(19) United States
(12) Patent Application Publication
(10) Pub. No.: US 2024/0302339 A1
(43) Pub. Date: Sep. 12, 2024

(54) OXYGEN CONSUMPTION ASSAY

(71) Applicant: PROMEGA CORPORATION, Madison, WI (US)

(72) Inventors: Ian MAROZAS, Madison, WI (US); Zhiyang ZENG, Madison, WI (US); Wenhui ZHOU, Madison, WI (US); Mike VALLEY, Madison, WI (US); Jolanta VIDUGIRIENE, Madison, WI (US); James CALI, Madison, WI (US); Tony LAUER, Madison, WI (US)

(21) Appl. No.: 18/600,390

(22) Filed: Mar. 8, 2024

Related U.S. Application Data
(60) Provisional application No. 63/489,551, filed on Mar. 10, 2023.

(51) Int. Cl. G01N 31/22; C12M 1/34; G01N 21/64

(52) U.S. Cl. CPC G01N 31/225; C12M 41/46; G01N 21/6408; G01N 2201/06193

(57) ABSTRACT

Provided herein are assays utilizing oxygen-sensitive fluorescent materials for the detection of oxygen. In particular, oxygen-sensitive fluorescent particles are provided for monitoring the oxygen consumption rate and metabolic fitness of living cells.

### CROSS-REFERENCE TO RELATED APPLICATION

The present invention claims priority to U.S. Provisional Patent Application Ser. No. 63/489,551, filed Mar. 10, 2023, which is hereby incorporated by reference in its entirety.

### FIELD

Provided herein are assays utilizing oxygen-sensitive fluorescent materials for the detection of oxygen. In particular, oxygen-sensitive fluorescent particles are provided for monitoring the oxygen consumption rate and metabolic fitness of living cells.

### BACKGROUND

Cells consume oxygen as they respirate, and the consumption rate of oxygen can be used to characterize the cellular metabolic phenotype. Currently, the most popular methods of oxygen consumption measurements require a standalone device and specialty equipment.

### SUMMARY

Provided herein are assays utilizing oxygen-sensitive fluorescent materials for the detection of oxygen. In particular, oxygen-sensitive fluorescent particles are provided for monitoring the oxygen consumption rate and metabolic fitness of living cells.

In some embodiments, provided herein are assays and components thereof capable of measuring the rate of cellular oxygen consumption on a standard microwell plate fluorescence measuring apparatus in under 30 minutes. The method converts a fluorescence signal from an oxygen sensitive dye into an oxygen concentration as a function of time. The method also is able to correct for variations of fluorescence due to temperature fluctuations of the indicator dye. Finally, the method is compatible with adherent cells, cells in suspension, and test compounds administered to the cells or media. Embodiments herein are capable of monitoring cellular fitness and using measurements to compare the metabolic fitness of cells, for example, comparing fitness before and after treatment with a chemical compound or comparing the fitness of two different cell lines. Cellular fitness can be defined as the physiological characteristics of cellular metabolism and mitochondrial function, for example, the maximal oxygen respiratory rate, the amount of proton leak, and the amount of non-mitochondrial oxygen consumption.

In some embodiments, provided herein are methods comprising: (a) placing an oxygen-sensitive composition into a sample to form a suspension of the composition within the sample; (b) exposing the oxygen-sensitive composition to light within an excitation spectrum of the oxygen-sensitive composition; (c) detecting light output within an emission spectrum from the oxygen-sensitive composition; and (d) determining a level of oxygen in the sample based on the light output. In some embodiments, steps (b)-(d) are repeated to monitor the level of oxygen over time. In some embodiments, methods further comprise (i) placing the oxygen-sensitive composition into a standard of known oxygenation level to form a suspension of the composition within the standard; (ii) exposing the oxygen-sensitive composition within the standard to light within an excitation spectrum of the oxygen-sensitive composition; (iii) detecting light output within an emission spectrum from the oxygen-sensitive composition within the standard; and (iv) determining a reference value for the light output from the oxygen-sensitive composition within the standard. In some embodiments, wherein steps (b)-(d) and (i)-(iv) are repeated to monitor the level of oxygen over time. In some embodiments, the suspension is contained within a microwell.

In some embodiments, provided herein are methods comprising: (a) contacting an oxygen-sensitive composition with a sample; (b) exposing the oxygen-sensitive composition to light within an excitation spectrum of the oxygen-sensitive composition; (c) detecting light output within an emission spectrum from the oxygen-sensitive composition; and (d) determining a level of oxygen in the sample based on the light output. In some embodiments, steps (b)-(d) are repeated to monitor the level of oxygen over time. In some embodiments, methods further comprise: (i) contacting the oxygen-sensitive composition with a standard of known oxygenation level; (ii) exposing the oxygen-sensitive composition within the standard to light within an excitation spectrum of the oxygen-sensitive composition; (iii) detecting light output within an emission spectrum from the oxygen-sensitive composition within the standard; and (iv) determining a reference value for the light output from the oxygen-sensitive composition within the standard. In some embodiments, steps (b)-(d) and (i)-(iv) are repeated to monitor the level of oxygen over time. In some embodiments, the oxygen-sensitive composition is adhered to a surface of a microwell. In some embodiments, the oxygen-sensitive composition is adhered to the bottom of a microwell.

In some embodiments, methods comprise a step of oxygenating and/or reoxygenating the sample. In some embodiments, oxygenating/reoxygenating the sample comprises stirring and/or shaking the sample. In some embodiments, oxygenating/reoxygenating the sample is performed before exposing the oxygen-sensitive composition to light.

In some embodiments, provided herein are methods comprising: (a) forming a hydrogel comprising an oxygen-sensitive composition with a sample; (b) exposing the oxygen-sensitive composition to light within an excitation spectrum of the oxygen-sensitive composition; (c) detecting light output within an emission spectrum from the oxygen-sensitive composition; and (d) determining a level of oxygen in the sample based on the light output. In some embodiments, steps (b)-(d) are repeated to monitor the level of oxygen over time. In some embodiments, methods further comprise: (i) forming a standard hydrogel comprising the oxygen-sensitive composition and a standard of known oxygenation level; (ii) exposing the oxygen-sensitive composition within the standard hydrogel to light within an excitation spectrum of the oxygen-sensitive composition; (iii) detecting light output within an emission spectrum from the oxygen-sensitive composition within the standard hydrogel; and (iv) determining a reference value for the light output from the oxygen-sensitive composition within the standard hydrogel. In some embodiments, steps (b)-(d) and (i)-(iv) are repeated to monitor the level of oxygen over time. In some embodiments, the hydrogel is contained within a microwell. forming a hydrogel comprises (i) contacting a pre-hydrogel liquid with the oxygen-sensitive composition and (ii) inducing hydrogel formation. In some embodiments, forming a hydrogel comprises exposure to temperature, pH, ions, light, or a small molecule inducer.

In some embodiments, determining the level of oxygen in the sample comprises comparing the light output to one or more reference values. In some embodiments, one of the reference values is obtained from light output of a deoxygenated standard. In some embodiments, one of the reference values is obtained from light output of an oxygenated standard. In some embodiments, one or more of the reference values is obtained from light output of standards of varying degrees of oxygenation.

In some embodiments, provided herein are methods to measure an oxygen consumption rate for a population of cells, comprising: (a) contacting an oxygen-free control, an oxygen-saturated control, and one or more test samples comprising the cells with an oxygen-sensitive composition; (b) measuring fluorescence intensities of the oxygen-sensitive composition in the controls and samples(s) over a period of time; (c) converting the fluorescent intensity of the sample(s) to oxygen concentration using (i) the Stern-Volmer equation and (ii) the fluorescent intensities of the controls, thereby calculating the oxygen consumption rate of the cells in the sample(s). In some embodiments, prior to step (b), the sample(s) are administered an agent that prevents mitochondrial activity, such that the oxygen consumption rate measured is the result of non-mitochondrial cellular respiration. In some embodiments, the agent that prevents mitochondrial activity is rotenone, oligomycin, or a combination thereof. In some embodiments, cell metabolic potential is assessed by subtracting non-mitochondria oxygen consumption from basal oxygen consumption. In some embodiments, prior to step (b), the sample(s) are administered an agent that stimulates respiration, and the oxygen consumption rate is measured. In some embodiments, the agent that stimulates respiration is carbonyl cyanide-p-trifluoromethoxyphenylhydrazone (FCCP). In some embodiments, the fluorescent intensity of the sample(s) to oxygen concentration is converted to a temperature-corrected oxygen concentration, therefore the oxygen consumption rate calculated is corrected for fluctuations in temperature.

In some embodiments, provided herein are methods to measure respiratory capacity, comprising: (i) measuring non-mitochondrial cellular respiration by contacting the sample(s) with an oxygen-sensitive composition and an agent or combination that prevents mitochondrial activity; (ii) further measuring the maximum oxygen consumption by contacting the sample(s) with an agent that stimulates mitochondria respiration; (iii) calculating respiratory capacity by subtracting non-mitochondria oxygen consumption from the maximum oxygen consumption.

In some embodiments, samples comprise T cells, B cells, or NK cells. In some embodiments, samples comprise embryonic stem cells, tissue-specific stem cells, mesenchymal stem cells, or induced pluripotent stem cells. In some embodiments, samples comprise adherent cell lines. In some embodiments, samples comprise suspension cell lines.

In some embodiments, measuring fluorescence intensities comprises: (i) exposing the oxygen-sensitive composition to light within an excitation spectrum of the oxygen-sensitive composition; (ii) detecting light output within an emission spectrum from the oxygen-sensitive composition. In some embodiments, fluorescence intensities are measured over a time period of 1 minute to 48 hours (e.g., multiple timepoints over the course of 1 minute, 2 minutes, 3 minutes, 4 minutes, 5 minutes, 10 minutes, 15 minutes, 20 minutes, 25 minutes, 30 minutes, 45 minutes, 1 hour, 2 hours, 3 hours, 4 hours, 5 hours, 6 hours 8 hours 12 hours, 16 hours, 26 hours, 36 hours, 48 hours, or ranges therebetween). In some embodiments, fluorescence intensities are measured every 1 second to 1 hour over a time period (e.g., every 1 second, 2 seconds, 3 seconds, 4 seconds, 5 seconds, 10 seconds, 15 seconds, 20 seconds, 30 seconds, 45 seconds, 1 minute, 2 minutes, 5 minutes, 10 minutes, 15 minutes, 20 minutes, 30 minutes, 45 minutes, 1 hour, or ranges therebetween).

In some embodiments, provided herein are oxygen detection systems comprising: (a) an oxygen-sensitive composition comprising an oxygen-sensitive fluorophore and a substrate; (b) a liquid sample comprising cells in an assay media; (c) an oxygenated standard; and (d) a deoxygenated standard. In some embodiments, the oxygenated standard comprises the assay media in the absence of cells. In some embodiments, the deoxygenated standard comprises glucose oxidase and glucose. In some embodiments, the deoxygenated standard comprises NaSO₃ and water. In some embodiments, oxygen detection systems herein further comprise a microwell plate, wherein at least a first well of the microwell plate comprises the liquid sample and the oxygen-sensitive composition, at least a second well of the microwell plate comprises the oxygenated standard and the oxygen-sensitive composition, and at least a third well of the microwell plate comprises the deoxygenated standard and the oxygen-sensitive composition. In some embodiments, the oxygen-sensitive compositions are adhered to a surface of the wells of the microwell plate. In some embodiments, the oxygen-sensitive compositions are adhered to the bottom of the wells of the microwell plate. In some embodiments, the oxygen-sensitive composition is suspended in the liquid sample, the oxygenated standard, and/or the deoxygenated standard. In some embodiments, the oxygen-sensitive composition is embedded in a 3D hydrogel with the liquid sample, the oxygenated standard, and/or the deoxygenated standard. In some embodiments, oxygen detection systems herein further comprise an instrument capable of detecting fluorescence. In some embodiments, the instrument is a plate reader.

In some embodiments of the methods and systems herein, the oxygen-sensitive composition comprises a substrate and an oxygen-sensitive fluorophore. In some embodiments of the methods and systems herein, the substrate is a particle. In some embodiments, the substrate is a spherical bead. In some embodiments, the particle is 5 nm to 10 μm in diameter, length, and/or width. In some embodiments, the particle is 250-600 nm in diameter, length, and/or width. In some embodiments, the substrate comprises a polymeric or inorganic material. In some embodiments, the substrate comprises polystyrene, polyacrylate, polyacrylic acid, polyacrylamide, polysiloxane, polyepoxide, polycarbonate, copolymers thereof, and functionalized polymers and copolymers thereof. In some embodiments, the substrate comprises polystyrene. In some embodiments, the substrate comprises carboxylate polystyrene particles. In some embodiments, the oxygen-sensitive fluorophore is adhered to, conjugated to, or impregnated within the substrate. In some embodiments, the oxygen-sensitive composition comprises a polymeric particle of 250-600 nm in diameter with an oxygen-sensitive fluorophore adhered thereto and/or impregnated therein.

In some embodiments of the methods and systems herein, the oxygen-sensitive fluorophore is a metal-ligand complex. In some embodiments, the oxygen-sensitive fluorophore is selected from ruthenium (II) complexes, iridium (III) complexes, osmium complexes, rhenium complexes, and metalloporphyrin complexes. In some embodiments, the oxygen-sensitive fluorophore is selected from tris(4,7-diphenyl-1,10-phenanthroline) ruthenium(II) chloride (Ru-dpp), Platinum octacthylporphyrin; platinum(II) 2,3,7,8,12,13,17,18-octacthyl-21H,23H-porphyrin (PtOEP), Palladium(II) octaethylporphine (PdOEP), Platinum(II)-5,10,15,20-tetrakis-(2,3,4,5,6-pentafluorphenyl)-porphyrin (PtTfPP), palladium(II)-5,10,15,20-tetrakis-(2,3,4,5,6-pentafluorphenyl)-porphyrin (PdTFPP), platinum(II) octaethylporphyrinketone (PtOEPK), palladium(II) octaethylporphyrinketone (PdOEPK), platinum(II) tetraphenyltetrabenzoporphyrin (PtTPTBP), meso-Tetraphenyl-tetrabenzoporphine Palladium Complex (PdTPTBP), platinum(II) tetraphenyltetranaphthoporphyrin (PtTPTNP), and palladium(II) tetraphenyltetranaphthoporphyrin (PdTPTNP). In some embodiments, the oxygen-sensitive fluorophore is a porphyrin-based metal complex. In some embodiments, the oxygen-sensitive fluorophore is PtTfPP.

In some embodiments of the methods and systems herein, the sample comprises cells and/or is a liquid sample. In some embodiments, the liquid sample comprises cell media.

In some embodiments of the methods and systems herein, the bottom of the microwell is transparent. In some embodiments, the opening of the microwell is sealed (e.g., with a foil cover, with a transparent cover, etc.). In some embodiments, the opening of the microwell is sealed with an O₂-impermeable cover.

### BRIEF DESCRIPTION OF THE DRAWINGS

FIG. 1. General Synthesis Scheme for the oxygen sensitive composition.

FIG. 2. The working principle of the oxygen consumption assay described herein for adherent and suspension cells.

FIG. 3. Effect of bead size on bead settling rate. Beads greater than or equal to 2 um settle in the assay window, and the settling phenomenon alters the fluorescence signal of the composition.

FIG. 4. The pH dependent surface composition of the beads affects particle-particle aggregation. Beads with an unfunctionalized surface aggregate due to hydrophobic interaction. Beads with a carboxylate surface aggregate at low pH due to hydrophobic interaction.

FIG. 5. The bulk density of the oxygen sensitive composition affects the measured oxygen consumption rate in live cells. A minimum of 0.02% wt/vol is required for maximal apparent oxygen consumption rate.

FIG. 6. Effect of assay volume on the measured oxygen consumption rate of adherent cells. Larger volumes result in a lower apparent oxygen consumption due to oxygen diffusion from the assay medium.

FIG. 7. Effect of assay volume on the measured oxygen consumption rate of suspension cells. Larger volumes result in a larger apparent oxygen consumption due to oxygen diffusion from the air-liquid interface.

FIG. 8. Effect of oxygen barrier on measured oxygen consumption rate. The presence of an oxygen barrier increases the apparent oxygen consumption rate by limiting oxygen diffusion from the atmosphere.

FIG. 9. Characteristic mitochondrial function determined from “metabolic stress test” assay. Various aspects of mitochondrial function can be ascertained by challenging the mitochondria with a specific series of mitochondrial poisons.

FIG. 10. General workflow of the oxygen consumption rate assay proposed herein.

FIG. 11. Effect of measurement interval on the apparent oxygen consumption rate. A shorter measurement interval (more frequent measurement) results in a lower apparent oxygen consumption rate, due to mixing effects during assay measurement.

FIG. 12. Metabolic potential of two different types of polarized macrophages.

FIG. 13. The oxygen consumption rate of cellular spheroids cultured in a three-dimensional synthetic matrix (Vitrogel matrix, TheWell Biosciences).

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies, or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a,” “an,” and “the” include plural reference unless the context clearly dictates otherwise. Thus, for example, reference to “a particle” is a reference to one or more particles and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3rd Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated hercin by reference.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “impregnated” refers to the condition of a material being filled with a component and/or having the component embedded within the material.

As used herein, the term “adhered to” refers to an interaction between a component and a substrate (e.g., via ionic bonding, hydrogen bonding, Van der walls forces, hydrophobic interactions, etc.) that results in the component being secured to the surface of the substrate.

As used herein , the term “conjugated” refers to the covalent linkage (e.g., directly or via a linker) of a component to a substrate.

As used herein, the term “metabolic potential” refers to a measurement of a cells ability to meet an energy demand via mitochondrial respiration and glycolysis. Metabolic potential can be calculated by:

$$( \frac{stressed OCR}{baseline OCR} ) * 100$$

As used herein, the terms “basal respiration,” “basal mitochondrial respiration,” “basal metabolic rate” (BMR), “baseline respiration” refer to the minimum rate of metabolism required by the mitochondria to support basic cellular functions. Basal mitochondrial respiration corresponds to the non-mitochondrial OCR subtracted from the basal cellular OCR; specifically, the OCR after oligomycin or rotenone administration subtracted from the OCR without drug treatment.

As used herein, the terms “maximal respiration,” “maximal mitochondrial respiration,” “maximal metabolic rate” (MMR), “stressed respiration” refers to the maximal rate of oxygen metabolism achievable by the mitochondria under chemically stressed conditions. Maximal mitochondrial respiration corresponds to the non-mitochondrial OCR subtracted from the stressed cellular OCR; specifically, the OCR after oligomycin or rotenone administration subtracted from the OCR after administration of a chemical stressor that increases mitochondrial respiration, for example, carbonyl cyanide-p-trifluoromethoxyphenylhydrazone (FCCP).

### DETAILED DESCRIPTION

Provided herein are assays utilizing oxygen-sensitive fluorescent materials for the detection of oxygen. In particular, oxygen-sensitive fluorescent particles are provided for monitoring the oxygen consumption rate and metabolic fitness of living cells.

In some embodiments, provided herein are biocompatible sensors (e.g., particles) that allow for measurement of oxygen content of a solution, and in particular, cellular oxygen consumption rate. In certain embodiments, the materials, systems, and assays herein allow for monitoring oxygen concentration and/or rate of change thereof in a standard format (e.g., 96-well plate, 384-well plate, etc.) using standard laboratory equipment (e.g., plate reader). Some embodiments herein provide functionalized materials (e.g., fluorescent particles) that can be used to detect the concentration of oxygen in a solution, hydrogel, on a surface, etc. (e.g., containing cells) over time.

In some embodiments, provided herein are polymer particles impregnated (or coated) with an oxygen-sensitive dye (e.g., porphyrin type). In some embodiments, the oxygen-sensitive particles are placed into a solution (e.g., media containing cells) to form a suspension. In other embodiments, a hydrogel is formed of a sample (e.g., cells and media) and the oxygen-sensitive particles Still further, the oxygen-sensitive particles may be adhered to a surface (e.g., a microwell) that is contacted by a sample (e.g., containing cells). Monitoring the fluorescence output of the oxygen-sensitive fluorophore allows detection/quantification of the oxygen content in the suspension/hydrogel/sample, monitoring of the cellular uptake of oxygen over time, and/or measuring the metabolic fitness of cells in the sample (e.g., in response to various stimuli or conditions).

In some embodiments, the materials that are impregnated (and/or coated) with an oxygen-sensitive dye (porphyrin type) are polymeric materials. In some embodiments, particles are impregnated (and/or coated) with an oxygen-sensitive dye. In some embodiments, particles of any suitable material may find use in embodiments herein. Suitable particles are biocompatible (e.g., non-toxic to cells), capable of remaining in suspension when placed into an aqueous solutions (e.g., cell media), capable of being embedded in a hydrogel, and/or capable of being impregnated and/or coated with a sufficient quantity of fluorophore, etc. Exemplary particle materials that find use in embodiments herein include polystyrene, polyacrylamide, Sepharose, silica, a polysaccharide polymer (e.g., agarose), polyesters, polyethylene, co-polymers thereof, etc. The materials (e.g., particles) may be surface functionalized or non-functionalized. Exemplary molecules for surface functionalization include carboxylate, amine, PEG, phosphate, sulfonate, sulfate, hydroxyl, epoxide. In some embodiments, the surface of the material (e.g., particle) is coated or functionalized with a polymer such as polyacrylic acid, etc. In some embodiments, particles exhibit a surface functionality such as to reduce or inhibit particle-particle aggregation. Such surface functionality includes covalent and non-covalent passive modifications. Exemplary covalent modifications to reduce aggregation include (i) charged molecules such as amine, substituted amine (e.g., morpholine), carboxylate, sulfonate, phosphate, and (ii) steric blockers such as, proteins (e.g., serum albumin, casein, pepticase, IgG), polymers (e.g., PEG, polysaccharide, polyester), graft polymers, and dendrimers. Exemplary passive modifications include proteins (bovine serum albumin, casein, pepticase, IgG), or non-ionic surfactants (e.g., Tween or Triton), or a combination of both. In some embodiments, particles used in the assays herein (e.g., impregnated with oxygen-sensitive dyes) are of any suitable size (e.g., 25 nm to 2 μm in diameter). In certain embodiments, particles are not less than 100 nm in diameter. In some embodiments, particles are not 2 μm or greater in diameter. In some embodiments, a population of particles has a mean diameter of 100 nm, 150 nm, 200 nm, 250 nm, 300 nm, 350 nm, 400 nm, 450 nm, 500 nm, 550 nm, 600 nm, 700 nm, 800 nm, 900 nm, 1200 nm, 1400 nm, 1600 nm, 1800 nm, 2000 nm, or ranges therebetween (e.g., 250-600 nm).

In some embodiments, provided herein are materials (e.g., particles) with oxygen-sensitive fluorophores embedded therein, coated thereon, and/or adhered thereto. In some embodiments, monitoring the fluorescence output of the oxygen-sensitive fluorophore allows detection/quantification of the oxygen content in a solution/sample in contact with the material (e.g., particles), monitoring of the cellular uptake/consumption of oxygen over time, and/or monitoring cellular/metabolic fitness. In some embodiments, the assays and materials herein comprise an oxygen-sensitive fluorophore (e.g., embedded within particles). In some embodiments, herein are oxygen-sensing materials (e.g., particles) comprising an oxygen-sensitive fluorophore embedded. In some embodiments, the oxygen-sensitive dye is adhered to the surface of a material (e.g., particle). In some embodiments, the oxygen-sensitive dye is a porphyrin dye. In some embodiments, the oxygen-sensitive fluorophore is selected from one of the following (wherein M=Pt(II) or M=Pd(II)):

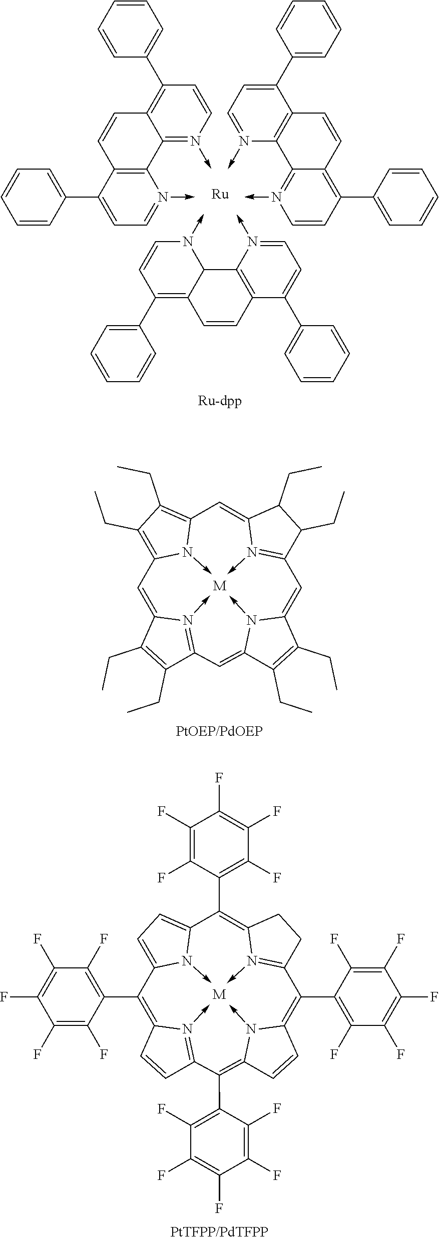

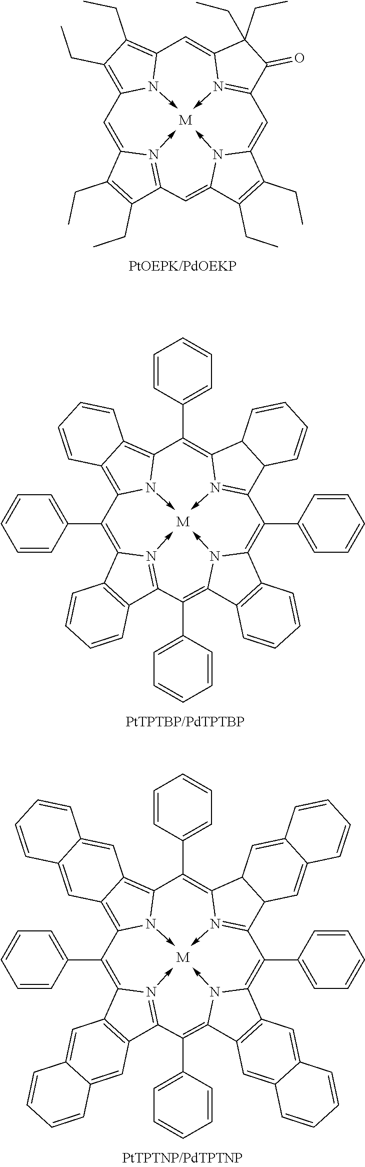

Other oxygen-sensitive fluorophores that are understood to those in the field are within the scope herein and may be included in the compositions (e.g., particles) described herein.

When the oxygen-sensitive fluorophores are brought to an excited state by exposure to light at an appropriate excitation wavelength, they relax to a ground state by emitting light at an emission wavelength. The intensity of the emitted light is dependent upon the oxygen concentration. Therefore, the signal emitted from the fluorophores is a function of oxygen concentration. Monitoring the signal of the fluorophores allows one to monitor the oxygen concentration of a sample in contact with the polysiloxane matrix material.

In some embodiments, the materials herein do not utilize (or contain) an oxygen-insensitive fluorophore, In other embodiments, some materials herein comprise an oxygen-insensitive fluorophore. In some embodiments, the oxygen-insensitive fluorophore is embedded within the material (e.g., particles). In some embodiments, the oxygen-insensitive fluorophore is adhered to the surface of the material (e.g., particles). In some embodiments, herein are oxygen-sensing materials (e.g., particles) comprising an oxygen-sensitive fluorophore and an oxygen-insensitive fluorophore, wherein the oxygen-sensitive fluorophore is used to monitor the oxygen concentration of a solution and the oxygen-insensitive fluorophore is used an oxygen-concentration-independent reference. Examples of oxygen-insensitive fluorophores that find use in embodiments herein include Coumarin-6, Nile blue chloride, tris(8-hydroxyquinolinato)aluminum (AlQ3), TAMRA, and fluorescein derivatives. In some embodiments, the materials provided comprise Coumarin-6 and PtTfPP. In other embodiments, control solutions (e.g., I₀ and I₁₀₀) are used as controls and an oxygen-insensitive fluorophore is not utilized (nor included on/within the materials (e.g., particles) herein.

In some embodiments, materials herein comprise a between 0.001 wt % and 2.5 wt % (e.g., 0.001 wt % to 2.5 wt %, 0.01 wt % to 2.4 wt %, 0.1 wt % to 2.3 wt %, 0.5 wt % to 2.2 wt %, 0.75 wt % to 2.1 wt %, 1 wt % to 2 wt %, or ranges therebetween) of oxygen-sensitive fluorophore in the material

$$( e . g . , ( \frac{mass of fluorophore}{total mass} ) * 100 ) .$$

For example, in some embodiments materials herein comprise 0.001 wt %, 0.005 wt %, 0.01 wt %, 0.02 wt %, 0.03 wt %, 0.04 wt %, 0.05 wt %, 0.06 wt %, 0.07 wt %, 0.08 wt %, 0.09 wt %, 0.1 wt %, 0.2 wt %, 0.3 wt %, 0.4 wt %, 0.5 wt %, 0.6 wt %, 0.7 wt %, 0.8 wt %, 0.9 wt %, 1.0 wt %, 1.1 wt %, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, or 2.5 wt % of oxygen-sensitive fluorophore in the material

$$( e . g . , ( \frac{mass of fluorophore}{total mass} ) * 100 ) .$$

For a dyed polymer particle density, the fluorophore weight percent can be estimated by extracting the dye into a known volume of THF, then comparing the absorption at 400 nm to a standard curve. In some embodiments, the absorption of the dye is directly measured in the particle without extraction.

In some embodiments, the oxygen-sensitive materials herein are added to a sample (test sample and/or control sample), and fluorescence measurements are made of the oxygen-sensitive materials within the sample. In some embodiments, the sample is a liquid or a hydrogel. In some embodiments, the sample (e.g., test sample) comprises cells. In some embodiments, the sample comprises cells in growth media. In some embodiments, the cells are in a liquid growth medium. In other embodiments, the cells and oxygen-sensitive materials (e.g., particles) are within a 3D growth media. Culture media may be any standard cell growth medium, preferably without phenol red and preferably without fetal bovine serum. In some embodiments, the assay media is at any suitable temperature (e.g., optimal temperature for cell growth, assay temperature, etc.). In some embodiments, the assay medium is warmed to the assay temperature (e.g., 37° C.) prior to the assay.

In some embodiments, the oxygen-sensitive materials are present in the sample at a density (mass of particles per volume of sample) od 0.001-1.0 w/v % (e.g., 0.001 w/v %, 0.002 w/v %, 0.005 w/v %, 0.010 w/v %, 0.02 w/v %, 0.05 w/v %, 0.10 w/v %, 0.2 w/v %, 0.5 w/v %, 1.0 w/v %, or ranges therebetween).

In some embodiments, the assays herein utilize an oxygen-depleted control or reference (I₀) and/or an oxygen-saturated control or reference (I₁₀₀). In some embodiments, the inclusion of a such references allows for calibration of the instrument used to detect fluorescence intensity. For example, the gain of the instrument (e.g., plate reader) is adjusted to accommodate the dynamic range of fluorescence intensity for these standards (thereby accommodating all possible fluorescence intensities for test samples). In some embodiments, the oxygen concentration within test samples is calculated based on one or both of the reference standards (e.g., I₀ and/or I₁₀₀). In some embodiments, the inclusion of I₀ and/or I₁₀₀ controls renders the assays agnostic the components of the samples, types of cells, type of media, assay conditions (e.g., temperature, pH, etc.).

In some embodiments, an instrument is utilized to measure fluorescence intensity of the test samples and/or control samples of the systems and assays described herein. Any instrument capable of exciting the fluorophores used in the systems and methods herein and detecting the emission of such fluorophores may find use in embodiments herein. In some embodiments, the methods and systems herein do not require specialized instruments but are instead compatible with standard fluorescence detectors, such as a plate reader. In some embodiments, an instrument (e.g., plate reader) is utilized that allows for rapid excitation of fluorophores and detection of fluorescence intensity in multiple wells of a microwell plate.

In some embodiments, assay conditions are such that an instrument (e.g., plate reader) is able to obtain sufficient fluorescence signal relative to machine noise. In some embodiments, a sufficient signal-to-noise ratio for embodiments herein is at least 20:1 (e.g., 20:1, 50:1, 100:1, 200:1, 300:1, 400:1, 500:1: or more). In some embodiments, the background subtracted I₀/I₁₀₀ ratio of the oxygen-sensitive material (e.g., particles) is between 2 and 5 (e.g., 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, and ranges therebetween) independent of the particle density in the measurement.

In some embodiments, provided herein are methods of detecting the oxygen concentration within a solution. In some embodiments, methods are provided for determining an absolute oxygen concentration, while in other embodiments, methods are provided for determining the oxygen concentration relative to a reference or control. In certain embodiments, methods herein allow for the oxygen concentration to be monitored over time, thereby providing methods of monitoring the rate of change of the oxygen concentration in a sample. In some embodiments, methods herein allow for monitoring the cellular fitness of a cell population under a test set of conditions (e.g., under cellular stress, in the presence of a toxin, drug, or stressor, under treatment conditions, etc.). In some embodiments, methods herein allow for monitoring characterize the different aspects of cellular metabolism and fitness of a cell population under a test set of conditions (e.g., under cellular stress, in the presence of a toxin, drug, or stressor, under treatment conditions, etc.). In particular embodiments, methods are provided for monitoring the rate of oxygen consumption and/or production by a system. For example, cells consume oxygen as they respirate; therefore, monitoring the rate of oxygen depletion in a system comprising cells provides a method to monitor cellular metabolism.

Oxygen acts as a quencher of fluorescence of various fluorophores (e.g., PtTFPP). When such fluorophores are incorporated into particles, the time-integrated photon quantity emitted by a particle, for a given dose of excitation light, increases with decreasing oxygen concentration, as described by the Stern-Volmer relationship. In addition to integrated phosphorescent light intensity, the oxygen concentration also affects the time constant of phosphorescence decay, where increasing oxygen levels decrease the time constant of phosphorescence decay. The time constant of phosphorescence decay is defined as the time required for the phosphorescence signal, excited by an infinitesimally short burst of excitation light, to decay to e-1 of the initial signal intensity. In some embodiments, the oxygen concentration is calculated in a phosphorescence decay experiment by measuring the phosphorescence intensity in a time resolved fluorescence experiment, or by calculating the decay constant in a dual-read time resolve fluorescence experiment. In some embodiments, measurements of integrated phosphorescence intensity are used to determine the oxygen concentration at a given time-point.

In some embodiments, the methods and systems find use with any suitable container (e.g., microwell, microcentrifuge tube, cuvette, etc.) and/or in a microfluidic device. In particular embodiments, microwell plates are utilized/provided in embodiments herein. In some embodiments, the systems and method herein are compatible with 96-well, 384-well, or other microplate formats. In some embodiments, the oxygen-sensitive materials described herein (e.g., particles) are suspended within a liquid sample within a microwell or other container. In some embodiments, the oxygen-sensitive materials described herein (e.g., particles) are adhered to a surface (e.g., bottom) of a microwell or other container. In some embodiments, the oxygen-sensitive materials described herein (e.g., particles) are suspended within a hydrogel within a microwell or other container. In some embodiments, cells are suspended within a liquid sample within a microwell or other container. In some embodiments, cells are adhered to a surface (e.g., bottom) of a microwell or other container. In some embodiments, cells are suspended within a hydrogel within a microwell or other container.

In some embodiments, systems and methods herein comprise/utilize one or more bioactive and/or biocompatible protein(s) and/or molecules to enhance the interaction properties between the particles herein, the surface(s) of a container (e.g., microwell), and/or cells. In some embodiments, the proteins/molecules are passively deposited onto the particles and/or surface of a microwell. In other embodiments, the proteins/molecules are covalently or non-covalently conjugated to the surface of a microwell. In some embodiments, proteins/molecules are conjugated to the surface of a microwell. In some embodiments, one or more bioactive extracellular matrix proteins are deposited/conjugated onto the surface of a microwell. Examples of suitable bioactive extracellular matrix proteins for use herein include type I collagen, type IV collagen, fibronectin, laminin, vitronectin, and Matrigel. In some embodiments, one or more biocompatible molecules are deposited/conjugated onto the surface of a microwell. An example of a suitable biocompatible molecule for use herein includes polydopamine.

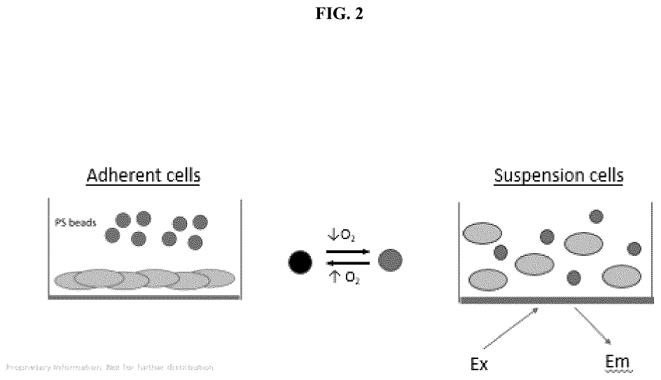

In some embodiments, an oxygen-sensitive particle solution is mixed with standard culture medium and administered to cells cultured on a microwell plate; the cells may be adherent or in suspension (FIG. 2). In some embodiments, the microwell plate is then sealed with an oxygen barrier to prevent back diffusion of oxygen from the atmosphere. An exemplary oxygen barrier is aluminum foil coated with an adhesive backing (e.g., SILVER SEAL). The SILVER SEAL type oxygen barrier allows test compounds to be added in sequence, and the Oxygen Consumption Rate (OCR) to be measured for a given sample multiple times, as the oxygen barrier is easy to remove and replace. Moreover, a SILVER SEAL type barrier leaves a small headspace of air, which re-oxygenates the assay medium immediately before assay measurement and provides more reproducible measurements from read to read, with larger values of apparent oxygen consumption. Oil-based oxygen barriers are superior at preventing oxygen diffusion from the atmosphere; however, they are slow to implement; they do not allow one to add additional test compounds to a sample because the barrier is impenetrable; and they do not allow for re-oxygenation of the system, which is important so that the initial oxygen concentration is the same for each measurement, particularly when adding compounds in sequence. Any suitable oxygen barriers, including SILVER SEAL type adhesive metallic barriers and oil-based barriers find use in embodiments herein.

In some embodiments, the materials within which oxygen-sensitive fluorophores are embedded (or upon which there are adhered) attenuate the light used to excite the fluorophores (or emitted from the fluorophores). In such embodiments, it may be the case that the strongest fluorescence signal, and therefore oxygen measurement, originates from the particles closest to the excitation light and/or emission detector. Experiments have shown that at 0.05 wt %, the excitation light does not penetrate further than 4.5 mm into the assay medium. Thus, to optimize the oxygen measurement signal for adherent cells, the excitation light comes from the bottom of the microwell plate, such that the strongest fluorescence signal, and therefore oxygen measurement, originates from the particles closest to cell samples (i.e., the bottom of the well). Moreover, the particles at the top of the sample reside at the air-liquid interface and are under the influence of oxygen back diffusion from the atmosphere. Consequently, the change of measured oxygen concentration is much smaller for measurements collected from the top of the well compared to the bottom of the well.

As described below in the EXPERIMENTAL section, the oxygen concentration in a sample can be measured using the materials described herein, by application of the Stern-Volmer equation, which relates the relative fluorescence intensity of the oxygen-sensitive and an oxygen-insensitive fluorophores to the concentration of the oxygen.

$$[ {O}_{2} ] ( t ) = \frac{\frac{{I}_{0}}{I ( t )} - 1}{\frac{{I}_{0}}{{I}_{sat}} - 1} * {[ {O}_{2} ]}_{sat}$$

I₀ is a reference value equal to the ratiometric fluorescence intensity of the oxygen-sensitive fluorophore over the oxygen-insensitive fluorophore in the absence of oxygen. Iₛₐₜ (aka I₁₀₀)is a reference value equal to the ratiometric fluorescence intensity of the oxygen-sensitive fluorophore over the oxygen-insensitive fluorophore at oxygen saturation. I(t) is a measured value equal to the ratiometric fluorescence intensity of the oxygen-sensitive fluorophore over the oxygen-insensitive fluorophore at time-point t. [O₂]ₛₐₜ is a reference value equal to the saturation concentration of oxygen. Using the Stern-Volmer equation, and having the necessary reference values in hand, allows a user to calculate the oxygen concentration at any time (t) in a sample or to monitor changes in oxygen concentration over a span of time.

In addition to oxygen, temperature also plays a role in the phosphorescence intensity of certain fluorophores (e.g., PtTFPP) when incorporated into particles. Increased temperature increases the frequency of molecular collisions between the fluorophore (e.g., PtTFPP) and oxygen quencher molecules. Consequently, increased temperatures result in reduced integrated phosphorescence intensity. In some embodiments, the effect of temperature on the oxygen-sensitive materials may be accounted for by measuring fluorescence intensity with the oxygen-saturated and oxygen depleted controls at each timepoint. In an exemplary workflow, a user dispenses the oxygen-sensitive materials (e.g., particles) into the sample and immediately loads the samples onto a plate reader device, which is set to 37° C. so the sample (and particles) warm to equilibrium. During the thermal equilibrium phase, the phosphorescence intensity of the particles will change, independent of oxygen concentration. Certain embodiments herein include internal controls that correct the calculated oxygen concentration for temperature fluctuations during thermal equilibration. In some embodiments, a preincubation step is performed to bring the samples to assay temperature (e.g., 37° C.) to reduce the effects of thermal fluctuations. However, the preincubation approach increases the assay time for the user.

In some embodiments, methods and compositions herein provide for monitoring the fitness of a cell. The OCR is measured using the compositions and methods herein in the absence or presence of agents (cell fitness agents (e.g., oligomycin, FCCP, rotenone, antimycin, etc.) that affect or more characteristics of respiration and/or cellular function. In some embodiments, various measures of cellular fitness are extracted by monitoring the OCR in the presence of the agents and comparing the OCR values. Basal cell respiration is measured by performing the methods herein to measure OCR in the absence of a cell fitness agent. Oligomycin impacts mitochondrial activity by inhibiting ATP production; the remaining oxygen is consumed by non-mitochondrial respiration and proton leak). FCCP stimulates max oxygen consumption; oxygen consumed is due to max respiration+non-mitochondrial respiration. Rotenone/antimycin prevents all mitochondrial activity. Remaining oxygen is consumed by non-mitochondrial activity.

### EXPERIMENTAL

### Example 1

### Exemplary Particle Synthesis

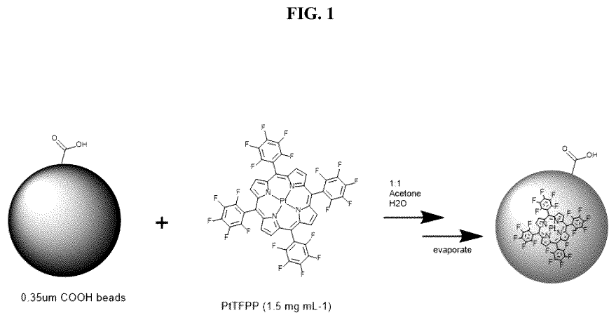

Exemplary oxygen-sensitive particles were synthesized by a dye absorption process into a polymer matrix (FIG. 1). PtTFPP (Platinum(II)-5,10,15,20-tetrakis-(2,3,4,5,6-pentafluorphenyl)-porphyrin), at a density of 0.150 mg mL−1 in tetrahydrofuran (THF), was added dropwise to an aqueous suspension (2.6 wt/vol %) of 350 nm polystyrene particles with carboxylate surface functionality, amine surface functionality, or non-functionalized. The solution was stirred for 4 hours, then THF was removed with rotary evaporation. The resulting suspension was filtered through a cell strainer, and then washed with 50% EtOH, 25% EtOH, and nanopure water. Washing is achieved by pelleting the particles with centrifugation at 7,000 rpm for 1 hour, then resuspending the particles in the washing medium with sonication and physical agitation. The dyed particles were of a uniform size of 350 nm, as confirmed by SEM imaging.

### Example 2

### Evaluation of Particle Characteristics

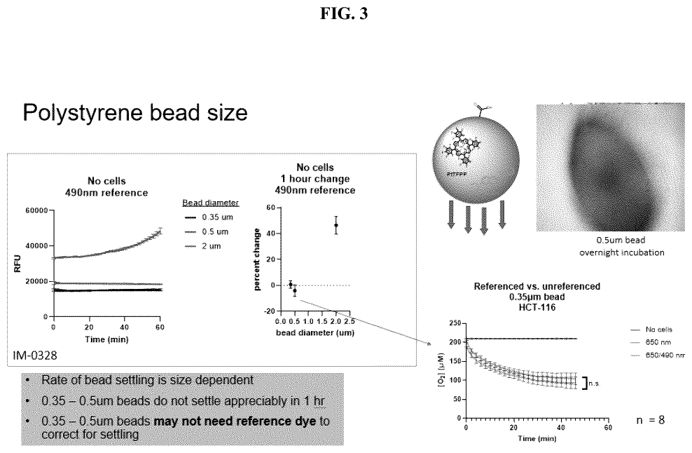

Experiments were conducted during development of embodiments herein to determine the effect of particle size (25 nm to 2 μm in diameter) within the assays/systems herein (FIG. 3). The experiments indicated that particles less than 100 nm in diameter were not viable with the synthesis scheme as the particles change size and become heterogeneous. Particles with a diameter of 2 μm or greater settle to the bottom of the assay plate, thereby altering the fluorescence signal during the assay window. Additional experiments were performed on particles sized 350-500 nm in diameter.

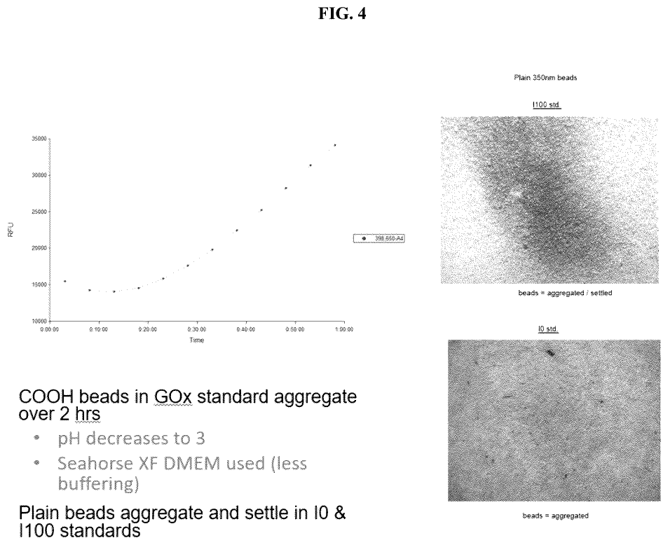

Experiments were conducted during development of embodiments herein to examine the effect of particle surface functionality. It was found that particles with a hydrophobic unfunctionalized polystyrene surface chemistry aggregate and settle to the bottom of the assay plate in the assay window (FIG. 4). It was also found that particles with either COOH or NH₂ surface chemistry do not tend to aggregate unless the pH or ionic strength is drastically changed from physiological conditions. Additionally, no difference in the cell OCR measurements was observed for particles of a COOH or NH₂ surface functionality, which suggests that aggregation is the most detrimental quality of the particles for OCR measurements.

### Example 3

### Exemplary Media Preparation

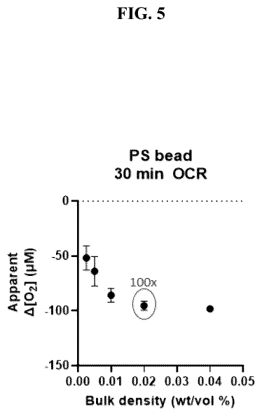

Exemplary assay medium was prepared by diluting the particles from 2.5% wt/vol to 0.025% wt/vol in culture medium, containing 10% vol/vol fetal bovine serum. Studies indicated that particle densities equal to or less than 0.05 wt/vol % yield lower apparent oxygen consumption rate measurements (FIG. 5). Particle densities greater than 0.05 wt % tend to aggregate within the 30-minute assay window. Experiments conducted during development of embodiments herein identified medium particle densities of 0.025 to 0.05 wt % as optimal.

### Example 4

### Exemplary Preparation of Internal Standards

Exemplary oxygenated (I₁₀₀) and deoxygenated (I₀) internal standards were prepared at a volume of 100 μL per well of a 96-well microplate and measured on the same plate as the samples. These standards correct for artifacts caused by temperature equilibration and consequently reduce assay duration for the user.

The I₁₀₀ standard is simply the same assay medium used for cell samples. I₀ standards are created by the addition of glucose oxidase GOx to a final concentration of 1 mg mL⁻¹. GOx consumes both glucose and oxygen to create D-glucono-1,5-lactone and hydrogen peroxide; consequently, deoxygenation of the lo standard is concomitant with presence of the glucose substrate. A sufficient concentration of glucose must be present in the lo standard solution to maintain the deoxygenated state, as oxygen back diffusion into the standard is constantly occurring. Experiments conducted during development of embodiments herein indicate that a concentration of 30 mM glucose is sufficient to maintain a hypoxic condition for at least 1-2 hours. Assay durations greater than 1-2 hours require refreshment of the lo standard or use of 1% wt/vol NaSO₃ in water as the Io standard.

### Example 5

### Adherent Cells

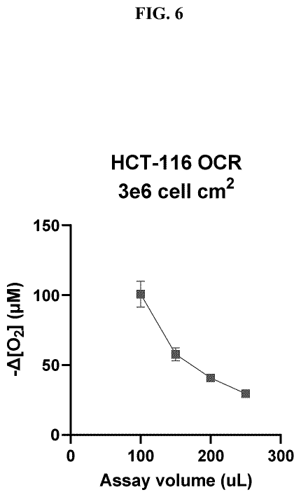

In an exemplary protocol, adherent cells are seeded the day prior to the oxygen consumption rate assay. On the day of the assay, the assay medium, along with any test compounds, is added to the cells at a volume of 100 μL per well for a 96-well plate or 40 μL for a 384-well plate. Experiments indicate that larger volumes of assay medium decrease the apparent OCR (FIG. 6). In some embodiments, a cell density titration is performed to determine the optimal density of cells that gives a reduction of at least 25% O₂ saturation in 30 minutes. In exemplary embodiments, 200e3-300e3 cells cm⁻² was sufficient seeding density of cells.

### Example 6

### Suspension Cells

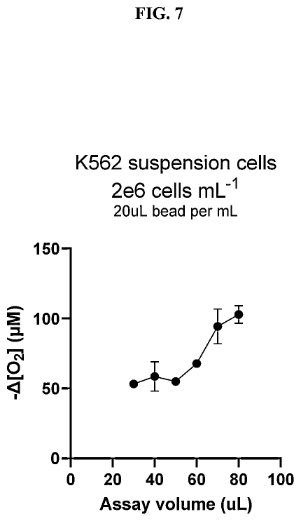

In an exemplary protocol, suspension cells are seeded the day of the oxygen consumption rate assay. The assay medium is combined with the cells along with any test compounds, and the mixture is dispensed at a volume of 200 μL per well for a 96-well plate or 80 μL for a 384-well plate. Smaller volumes of assay medium decreased the apparent OCR (FIG. 7). In some embodiments, a cell density titration is performed to determine the optimal density of cells that gives a reduction of at least 25% O₂ in 30 minutes. In exemplary embodiments, 2e6-3e6 cells mL⁻² is a sufficient seeding density of cells.

### Example 7

### OCR Measurement on Plate Reader

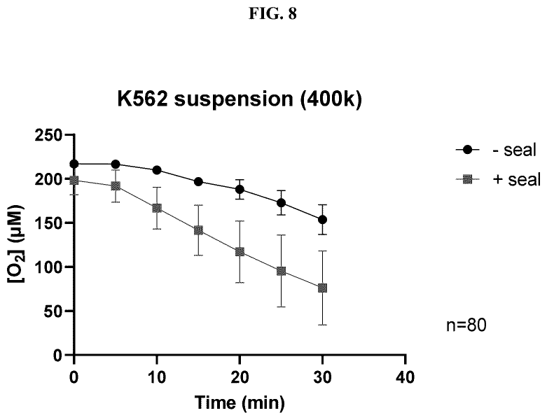

In an exemplary protocol, after adding the assay medium to the cells, an oxygen barrier is placed on top of the microwell plate. In experiments conducted during development of embodiments herein, a foil-based covering with an adhesive backing was used. The assay has been performed without an oxygen barrier, but oxygen exchange with the air alters the results (FIG. 8). Plates are typically read from the bottom; however, reading the plate from above is also feasible (e.g., with an open top or transparent barrier (e.g., oil or clear film).

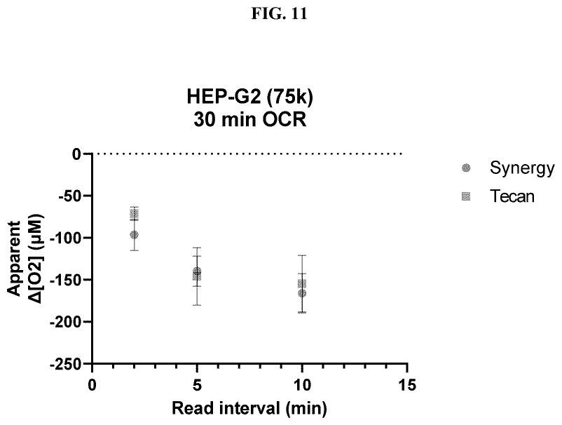

Plates are typically read at an interval of 2-30 minutes between measurements for both 96-well plates and 384-well plates. The plate-reading interval influences the measured change in oxygen concentration (“apparent Δ[O₂]”). Reading the plate more frequently at 2 minutes per read yields a lower apparent change in the oxygen concentration compared to reading the plate less frequently at 10 minutes per read (FIG. 11). The plate-reading interval affects the measured oxygen concentration because the plate reader mixes oxygen into the medium as it moves the plate from position to position.

### Example 8

### Metabolic Fitness Testing

Experiments were conducted during development of embodiments herein to measure the metabolic fitness of cell samples by testing oxygen consumption rate in the presence of a series of drugs that target certain mitochondrial functions. The optimal concentration of FCCP must first be determined before executing the “metabolic stress test.” The “metabolic stress test” is executed by first measuring the untreated basal OCR, then removing the oxygen barrier, adding oligomycin, replacing the oxygen barrier, shaking the plate to reoxygenate the assay media, then measuring the O₂ consumption linked to ATP generation; the process is repeated for FCCP and rotenone treatment (FIG. 8). Additional media volume is also added to I₀ and I₁₀₀ standards to account for the volume added to the samples.

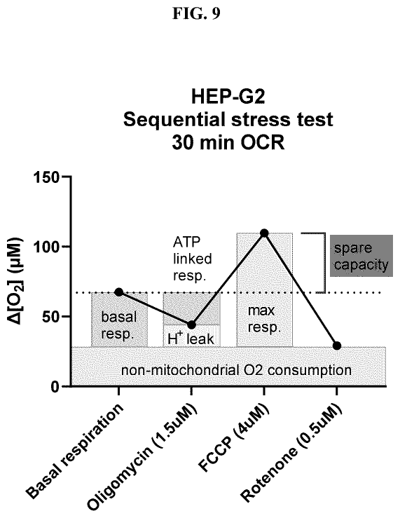

From the metabolic stress test OCRs, the difference of OCR between treatments was calculated and used to estimate the amount of oxygen consumed in various metabolic processes. The metabolic processes that contribute to metabolic fitness are as follows (FIG. 9).
1. non mitochondrial O₂ consumption [rotenone]
2. maximal respiration [FCCP-rotenone]
3. spare capacity [FCCP-untreated]
4. basal mitochondrial respiration [untreated-rotenone]
5. ATP linked respiration [untreated-oligomycin]
6. proton leak [oligomycin-rotenone]

### Example 9

### Exemplary Pre-Assay Protocol

The following is an exemplary protocol useful for certain embodiments of the compositions and methods described herein. Alternatives and modifications to the following, consistent with the disclosure herein are within the scope of embodiments herein. Oxygen-sensitive particle suspension is stored at 4° C. until use. The particles settle during storage. Prior to use, the particles are mixed by gentle inversion 5-10 times.

For measurements with the oxygen consumption assay, a fluorescence intensity microplate reader is used that is capable of read-out from the bottom of a plate. A single filer pair is used for measurements with the oxygen consumption assay:

| | Excitation | Emission |
|---|---|---|
| Filter pair | 400 nm | 650 nm |
| Bandwidth | 20 nm | 20 nm |

For some plate readers (e.g., Tecan Spark), the advanced settings on the “standard read mode” can be altered to yield better performance with phosphorescent dyes. Namely, the measurement data collection delay (integration start) can be set to 0 μs, and the measurement collection time (integration time) can be set to >500 μs. Time resolved fluorescence mode (TRF mode) can be used with the assay, and the aforementioned delay and integration times can be used.

### Step 1: Autogain

Gain mode: standard (not extended)
Scale to high wells: select Io wells (positive control, containing Na₂SO₃ or Glucose Oxidase)
Scale value: 80% of maximal RFU at 650 nm

### Step 2: Shake 3 Minutes (Fast)

### Step 3: Kinetic Read

Gain mode: standard (not extended)
Gain value: Use gain value determined in Step 1
Kinetic read interval: 5 minutes
Measurement delay after plate movement: 100 msec
Measurement collection time (integration time): >500 μs (very important)
Measurement data collection delay (integration start): 0 μs (very important)
Measurements per well: 30
Delay between measurements: 1 msec

### Example 10

### Exemplary Assay Protocol for Adherent Cells

### Day 1

Seed cells at optimal cell density (determined prior to performing experiments with drug treatment).

### Day 2

Turn on plate reader and warm environmental chamber to 37° C. for 1 hour
Warm culture medium to 37° C.
   Growth medium without phenol red recommended, but not required
Prepare I₀ calibration standard solutions (positive control), either
   10mL deionized water +100 mg sodium sulfite (recommended)
      Should be made fresh before each assay. For convenience, 100 mg sodium sulfite may be aliquoted into a 10 mL conical tube
      Leave little headspace in vessel. Close the vessel and shake it lightly for approximately one minute to dissolve Na₂SO₃ and to ensure that the water is oxygen-free. Vessel is kept closed to minimize oxygen ingress. The shelf life of I₀ calibration standard is typically about 8 hours provided that the vessel has been closed.
   500 μL of 10× Glucose oxidase in PBS (10 mg mL⁻¹).
      This 10× stock can be aliquoted and frozen
      Immediately before assay, the 10× stock is diluted to 1× in +glucose DMEM
      I₀ calibration standard made by this method can prepared by adding 10× glucose oxidase stock 1:10 directly into I₀ calibration standard wells. I₀ calibration standard wells made by this method are typically oxygen free for only about 1 hour.
Assay medium: O₂ particle suspension combined with drug compound. 10× cell suspension and 10× drug to be added to assay medium.
   Example for full plate (96 wells)
      17.8 mL-phenol+glucose DMEM
      200 μL 100× O₂ particle suspension
   Growth medium without phenol red recommended, but not required
   Growth medium contains glucose
Drug medium: Growth medium containing compound of interest (10× concentration) to each well. 20 μL to be added per well
   Example for full plate (80 wells) treated with 20 μL 10× compound A (2 mL final volume).
      1,800 μL-phenol DMEM+10% v/v FBS
      200 μL 10× compound A

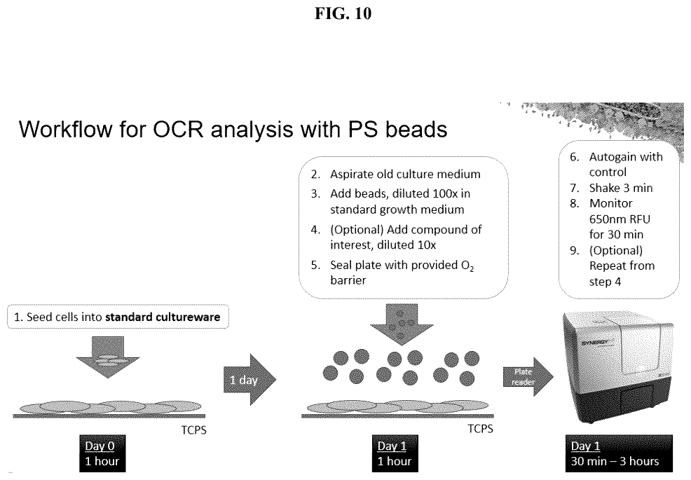

An exemplary oxygen consumption assay workflow is depicted in FIG. 10 and described below:

### Assay Instructions (Single Drug Addition)

### Day 1:

1. Seed cells at appropriate seeding density (e.g., a seeding density of 50,000-100,000 cells per well of a 96-well microplate (approximately 1.5×10⁵-3×10⁵ cells cm⁻²))
2. Allow cells to rest for 15-30 minutes at room temperature in a sterile workspace.
3. Transfer microplate to a cell culture incubator.

### Day 2:

1. Fill I₀ & I₁₀₀ calibration standard wells (positive and negative control wells, respectfully)
   a. I₀ calibration standard
      Glucose oxidase method: Mix 1620 μL assay medium (medium must contain glucose), and 180 μL 10× Glucose oxidase stock. Add 200 μL of this mixture to each I₀ calibration well. Make this I₀ calibration standard fresh for each measurement.
   b. I₁₀₀ calibration standard
      i. Mix 1620 μL assay medium and 180 μL PBS. Add 200 μL of this mixture to each I₁₀₀ calibration well.
2. Aspirate old growth medium from cells
3. For experiments without drug treatment (e.g., cell density titration), add 180 μL assay medium (above) to each well, then add 20 μL growth medium to each well. Alternatively, dilute assay medium 9:1 with growth medium and add 200 μL to each well.
4. For experiments with drug treatment, add 180 μL assay medium (above) to each well, then add 20 μL drug medium to each well. Alternatively, dilute assay medium 9:1 with drug medium and add 200 μL to each well. To minimize edge effects, treatment groups can be arranged by columns or squares instead of rows.
5. Seal plate with adhesive foil (Greiner cat #676090).
6. Immediately move plate to plate reader and initiate kinetic read

### Assay Instructions (Sequential Drug Addition)

### Day 1:

1. Seed cells at appropriate seeding density (e.g., seeding density of 50,000-100,000 cells per well of a 96-well microplate (approximately 1.5×10⁵-3×10⁵ cells cm⁻²))
2. Allow cells to rest for 15-30 minutes at room temperature in a sterile workspace.
3. Transfer microplate to a cell culture incubator.

### Day 2:

1. Fill I₀ & I₁₀₀ calibration standard wells (positive and negative control wells, respectfully)
   a. I₀ calibration standard
      i. Sodium sulfite method: Mix 1980 μL 1% sodium sulfite solution and 20 μL O₂ 100× particle suspension. Add 100 μL of this mixture to each I₀ well.
      ii. Glucose oxidase method: Mix 1620 μL assay medium (medium must contain glucose), and 180 μL 10× Glucose oxidase stock. Add 200 μL of this mixture to each I₀ calibration well. Make this I₀ calibration standard fresh for each measurement.
   b. I₁₀₀ calibration standard
      i. Mix 1620 μL assay medium and 180 μL PBS. Add 200 μL of this mixture to each I₁₀₀ calibration well.
2. Aspirate old growth medium from cells
3. Add 180 μL assay medium (above) to each well.
4. Seal plate with adhesive foil (Greiner cat #676090).
5. Immediately move plate to plate reader and initiate kinetic read with.
6. Gently peel back adhesive foil.
7. Add 20 μL drug medium containing compound of interest (10× concentration) to each well. To minimize edge effects, treatment groups can be arranged by columns or squares instead of rows.
8. Add 20 μL growth medium to I₁₀₀ calibration standard wells.
9. Add 20 μL 1% sodium sulfite solution I₀ calibration standard wells.
10. Re-seal plate with adhesive foil.
11. Immediately move plate to plate reader and initiate kinetic read.
12. Repeat steps 6-11, adding additional volumes of 10× treatment medium at steps 7-9 to account for larger initial volume (e.g., add 22 μL 10× stock to a well that now contains 200 μL).

### Example 11

### Exemplary Assay Protocol for Cells in Suspension

The following is an exemplary protocol useful for certain embodiments of the compositions and methods described herein. Alternatives and modifications to the following, consistent with the disclosure herein are within the scope of embodiments herein.
Turn on plate reader and warm environmental chamber to 37° C. for 1 hour
Warm culture medium to 37° C.
   Growth medium without phenol red recommended, but not required
Prepare Io calibration standard solutions (positive control)
   10 mL deionized water +100 mg sodium sulfite
      Must be made fresh before each assay. For convenience, 100 mg sodium sulfite may be aliquoted into a 10 mL conical tube
      Leave little headspace in vessel. Close the vessel and shake it lightly for approximately one minute to dissolve Na₂SO₃ and to ensure that the water is oxygen-free. Keep the vessel closed to minimize oxygen ingress. The shelf life of Io calibration standard is about 8 hours provided that the vessel has been closed.
   500 μL of 10× Glucose oxidase in PBS (10 mg mL⁻¹).
      This 10× stock can be aliquoted and frozen
      Immediately before assay, the 10× stock is diluted to 1× in +glucose DMEM
      I₀ calibration standard made by this method can prepared by adding 10× glucose oxidase stock 1:10 directly into I₀ calibration standard wells. I₀ calibration standard wells made by this method are typically oxygen free for about 1 hour.
Assay medium: O₂ particle suspension combined with drug compound. 10× cell suspension and 10× drug to be added to assay medium.
   Example for full plate (96 wells)
      15.8 mL-phenol+glucose DMEM
      200 μL 100× O₂ particle suspension
   Growth medium without phenol red recommended, but not required
   Growth medium must contain glucose
Drug medium: Growth medium containing compound of interest (10× concentration) to each well. 20 uL to be added per well
   Example for full plate (80 wells) treated with 20 uL 10× compound A (2 mL final volume).
      1,800 uL-phenol DMEM+10% v/v FBS
      200 uL 10× compound A
         Assay instructions (single drug addition)
1. Fill I₀ & I₁₀₀ calibration standard wells (positive and negative control wells, respectfully)
   a. I₀ calibration standard
      i. Sodium sulfite method: Mix 1980 μL 1% sodium sulfite solution and 20 uL O₂ 100× particle suspension. Add 100 μL of this mixture to each I₀ well.
      ii. Glucose oxidase method: Mix 1440 uL assay medium (medium must contain glucose), 180 uL growth medium, and 180 uL 10× Glucose oxidase stock. Add 200 uL of this mixture to each Io calibration well. Make this Io calibration standard fresh for each measurement.
   b. I₁₀₀ calibration standard
      i. Mix 1440 uL assay medium and 360 uL PBS. Add 200 uL of this mixture to each I₁₀₀ calibration well.
2. Dilute 10× cells in assay medium (above) (8:1 assay medium to cell suspension) and add 180 μL to each well.
3. Add 20 μL drug medium to treated wells.
   a. Alternatively, dilute cell suspension in assay medium (above) (9:1 cell suspension to drug medium) and add 200 μL to each well.
4. Add 20 μL growth medium to untreated control wells.
   a. Alternatively, dilute cell suspension in assay medium (above) (9:1 cell suspension to growth medium) and add 200 μL to each well.
5. Seal plate with adhesive foil (Greiner cat #676090).
6. Immediately move plate to plate reader and initiate kinetic read.

### Assay Instructions (Sequential Drug Addition)

1. Fill I₀ & I₁₀₀ calibration standard wells (positive and negative control wells, respectfully)
   a. I₀ calibration standard
      Glucose oxidase method: Mix 1440 uL assay medium (medium must contain glucose!), and 180 uL 10× Glucose oxidase stock. Add 180 uL of this mixture to each I₀ calibration well. Make this I₀ calibration standard fresh for each measurement.
   b. I₁₀₀ calibration standard
      i. Mix 1440 uL assay medium and 180 uL PBS. Add 180 uL of this mixture to each I₁₀₀ calibration well.
2. Dilute cell suspension in assay medium (above) (8:1 assay medium:cell suspension) and add 180 μL to each well.
3. Seal plate with adhesive foil (Greiner cat #676090).
4. Immediately move plate to plate reader and initiate kinetic read.
5. Gently peel back adhesive foil.
6. Add 20 μL growth medium containing compound of interest (10× concentration) to each well.
7. Add 20 μL growth medium to I₁₀₀ calibration standard wells.
8. Add 20 μL 1% sodium sulfite solution I₀ calibration standard wells.
9. Re-seal plate with adhesive foil
10. Immediately move plate to plate reader and initiate kinetic read.
11. Repeat steps 6-11, adding additional volumes of 10× treatment medium at steps 6-8 to account for larger initial volume (e.g., add 22 μL 10× stock to a well that now contains 200 μL).

### Example 12

### Metabolic Potential of Differentiated Macrophages

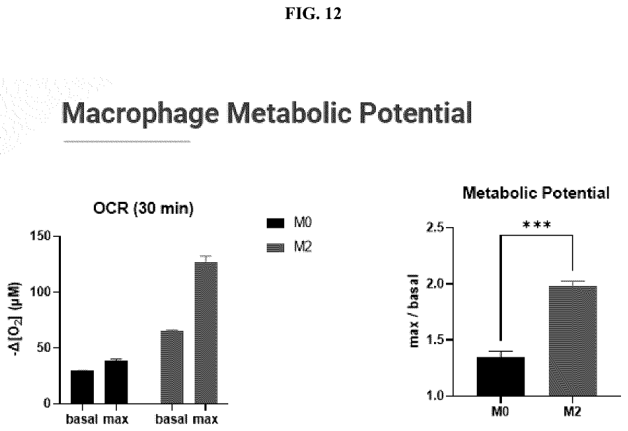

Experiments were conducted during development of embodiments herein to compare the metabolic potential of primary monocytes differentiated into unpolarized M0 macrophages and polarized M2 macrophages using oxygen-sensitive beads (prepared as in Example 1). Primary monocytes were cultured and differentiated into either M0 or M2 macrophages. Upon differentiation, the oxygen-sensitive beads were introduced to the culture medium to measure the basal OCR, followed by treatment with BAM15 to measure the maximal respiration rate. The metabolic potential was assessed by calculating the quotient of the maximum OCR to the basal OCR for both M0 and M2 macrophages. The M2 macrophages exhibited a higher metabolic potential compared to MO macrophages, as evidenced by a larger quotient of maximum to basal OCR (See FIG. 12). This observation aligns with existing literature, underscoring the beads' capability to accurately reflect the metabolic states of differentially polarized macrophages.

### Example 13

### Oxygen Consumption Rate of Spheroids in a Three-Dimensional Matrix

Experiments were conducted during development of embodiments herein to demonstrate the efficacy and versatility of oxygen-sensitive particles (prepared as in Example 1) for measuring the oxygen consumption rate (OCR) in a three-dimensional cell culture model and to determine the limits of this assay with respect to the parameters of three-dimensional cell culture. Experiments were conducted during development of embodiments herein to establish a correlation between cell spheroid density, OCR, and a previously validated assay of metabolic activity.

### Cell Culture and Spheroid Formation

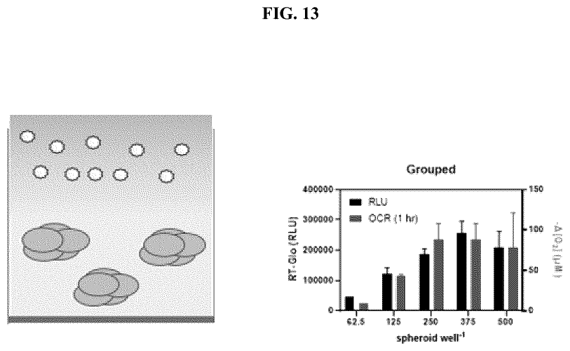

HCT-116 cell spheroids were prepared by seeding approximately 600 cells per spheroid in a Corning Elplasia plate and harvesting the spheroids after two days. The spheroids were encapsulated within a three-dimensional matrix (Vitrogel, TheWell Biosciences; FIG. 13, left panel) at increasing numbers of spheroids per well. This approach aimed to simulate a range of conditions with varying degrees of oxygen demand.

### Encapsulation and Addition of Oxygen-Sensitive Beads

The oxygen-sensitive beads were added to the overlying culture medium of the encapsulated spheroids. Despite the oxygen-sensitive beads being located in a different compartment relative to the spheroids, they still allow for precise measurement of the OCR by detecting changes in the medium's oxygen levels as oxygen is drawn from the medium into the spheroids.

### Oxygen Consumption Rate Measurement

OCR measurements were taken post-addition of the oxygen-sensitive beads. The experiment focused on the correlation between the spheroid density and the oxygen consumption rate.

### Metabolic Activity Assay

Concurrently, the RT-Glo MT assay was employed to measure the overall metabolic activity within the wells containing the spheroids. This assay provides a luminescent readout indicative of the metabolic activity, serving as a parallel measure to the OCR.

### Results

A direct correlation was observed between the spheroid density and the OCR, with higher densities showing significantly increased OCR (FIG. 13, right panel). This finding indicates that a greater number of spheroids, and consequently higher cell densities, consume more oxygen. The results from the OCR measurements demonstrated a strong correlation with the RT-Glo MT assay outcomes. This correlation further validates the use of the oxygen-sensitive dye encapsulated beads for accurately measuring metabolic activity in cell cultures, aligning with established metabolic assays.

These results underscore the utility of the oxygen-sensitive dye encapsulated beads in measuring oxygen consumption rates in a dynamic and three-dimensional cell culture environment. The ability to correlate OCR with cell density and metabolic activity, as evidenced by the strong alignment with the RT-Glo MT assay results, highlights the precision and reliability of this method for assessing cellular metabolism.

### REFERENCES

The following references are herein incorporated by reference in their entireties
Mercier-Letondal et al. J Transl Med (2021) 19:21.
Prutton et al. Free Radical Biology and Medicine 196 (2023) 11-21.
Read et al. Colloids and Surfaces B: Biointerfaces 171 (2018) 197-204.
Wang et al. Nature Communications (2021) 12:5846.

1. A method comprising:
(a) placing an oxygen-sensitive composition into a sample to form a suspension of the composition within the sample;
(b) exposing the oxygen-sensitive composition to light within an excitation spectrum of the oxygen-sensitive composition;
(c) detecting light output within an emission spectrum from the oxygen-sensitive composition; and
(d) determining a level of oxygen in the sample based on the light output.

2. A method comprising:
(a) contacting an oxygen-sensitive composition with a sample;
(b) exposing the oxygen-sensitive composition to light within an excitation spectrum of the oxygen-sensitive composition;
(c) detecting light output within an emission spectrum from the oxygen-sensitive composition; and
(d) determining a level of oxygen in the sample based on the light output.

3. The method of claim 1 or 2, further comprising a step of reoxygenating the sample.

4. The method of claim 3, wherein reoxygenating the sample comprises shaking the sample.

5. The method of claim 3, wherein reoxygenating the sample is performed before exposing the oxygen-sensitive composition to light.

6. A method comprising:
(a) forming a hydrogel comprising an oxygen-sensitive composition with a sample;
(b) exposing the oxygen-sensitive composition to light within an excitation spectrum of the oxygen-sensitive composition;
(c) detecting light output within an emission spectrum from the oxygen-sensitive composition; and
(d) determining a level of oxygen in the sample based on the light output.

7. The method of claim 6, wherein forming a hydrogel comprises (i) contacting a pre-hydrogel liquid with the oxygen-sensitive composition and (ii) inducing hydrogel formation.

8. A method to measure an oxygen consumption rate for a population of cells, comprising:
(a) contacting an oxygen-sensitive composition with an oxygen-free control, an oxygen-saturated control, and one or more test samples comprising the cells;
(b) measuring fluorescence intensities of the oxygen-sensitive composition in the controls and samples(s) over a period of time;
(c) converting the fluorescent intensity of the sample(s) to oxygen concentration using (i) the stern-volume equation and (ii) the fluorescent intensities of the controls, thereby calculating the oxygen consumption rate of the cells in the sample(s).

9. The method of claim 8, wherein the fluorescent intensity of the sample(s) to oxygen concentration is converted to a temperature-corrected oxygen concentration, therefore the oxygen consumption rate calculated is corrected for fluctuations in temperature.

10. The method of one of claims 1-9, wherein the oxygen-sensitive composition comprises a substrate and an oxygen-sensitive fluorophore.

11. The method of claim 10, wherein the substrate is a particle.

12. The method of claim 11, wherein the particle is a spherical bead.

13. The method of claim 11, wherein the particle is 5 nm to 10 μm in diameter, length, and/or width.

14. The method of claim 13, wherein the particle is 250-600 nm in diameter, length, and/or width.

15. The method of claim 10, wherein the substrate comprises a polymeric or inorganic material.

16. The method of claim 15, wherein the substrate comprises polystyrene, polyacrylate, polyacrylic acid, polyacrylamide, polysiloxane, polyepoxide, polycarbonate, copolymers thereof, and functionalized polymers and copolymers thereof.

17. The method of claim 16, wherein the substrate comprises polystyrene.

18. The method of claim 17, wherein the substrate comprises carboxylate polystyrene particles.

19. The method of claim 17, wherein the substrate comprises amine polystyrene particles.

20. The method of one of claims 10-19, wherein the substrate exhibits a surface functionality of sufficient density such as to reduce or inhibit particle-particle aggregation.

21. The method of claim 20, wherein the surface functionality to reduce particle-particle aggregation comprises one or more covalent modifications to the substrate.

22. The method of claim 21, wherein the one or more covalent modifications to reduce aggregation are selected from amine, substituted amine (e.g., morpholine), carboxylate, sulfonate, and phosphonate.

23. The method of claim 21, wherein the one or more covalent modifications to reduce aggregation comprise one or more steric blockers.

24. The method of claim 23, wherein the one or more steric blockers are selected from a protein, a peptide, a polysaccharide, a polymer, a graft polymer, a dendrimer, and combinations thereof.

25. The method of claim 24 , wherein the one or more steric blockers are selected from serum albumin, casein, pepticase, IgG, PEG, dextran, a polyester, and combinations thereof.

26. The method of claim 20, wherein the surface functionality to reduce particle-particle aggregation is non-covalently or passively adsorbed to the substrate.

27. The method of claim 26, wherein the surface functionality is a protein, a peptide, a polysaccharide, a polymer, a graft polymer, a dendrimer, an ionic surfactant, a non-ionic surfactant, or a combination thereof.

28. The method of claim 27, wherein the surface functionality comprises one or more of serum albumin, casein, pepticase, dextran, IgG, Tween, Triton, or combinations thereof.

29. The method of one of claims 10-28, wherein the oxygen-sensitive composition is stored as a suspension in an aqueous medium.

30. The method of claim 29, wherein the oxygen-sensitive composition is suspended at a density of 0.001-3% wt/vol.

31. The method of claim 29 or 30, wherein the oxygen-sensitive composition is suspended in an aqueous medium containing blocking agents to reduce particle-particle aggregation.

32. The method of claim 31, wherein blocking agents to reduce particle-particle aggregation comprises proteins and/or non-ionic surfactants.

33. The method of claim 32, wherein the proteins and/or non-ionic surfactants are selected from bovine serum albumin, casein, pepticase, IgG, Tween, Triton, and combinations thereof.

34. The method of any one of claims 29-33, wherein the aqueous medium contains up to 20% ethanol.

35. The method of any one of claims 29-34, wherein the aqueous medium contains an antimicrobial agent.

36. The method of claim 35, wherein the antimicrobial agent is selected from sodium azide, phenol, and phenol derivatives.

37. The method of claim 10, wherein the oxygen-sensitive fluorophore is adhered to the surface of the substrate.

38. The method of claim 10, wherein the oxygen-sensitive fluorophore is conjugated to the substrate.

39. The method of claim 10, wherein the substrate is impregnated with the oxygen-sensitive fluorophore.

40. The method of claim 10, wherein the oxygen-sensitive composition comprises a polymeric particle 5 nm to 10 μm in diameter with an oxygen-sensitive fluorophore adhered thereto and/or impregnated therein.

41. The method of claim 40, wherein the oxygen-sensitive composition comprises a polymeric particle of 250-600 nm in diameter with an oxygen-sensitive fluorophore adhered thereto and/or impregnated therein.

42. The method of one of claims 10-41, wherein the oxygen-sensitive fluorophore is selected from metal-ligand complexes.

43. The method of claim 42, wherein the oxygen-sensitive fluorophore is selected from ruthenium (II) complexes, iridium (III) complexes, osmium complexes, rhenium complexes, and metalloporphyrin complexes.

44. The method of claim 43, wherein the oxygen-sensitive fluorophore is selected from tris(4,7-diphenyl-1,10-phenanthroline) ruthenium(II) chloride (Ru-dpp), Platinum octaethylporphyrin; platinum(II) 2,3,7,8,12,13,17,18-octaethyl-21H,23H-porphyrin (PtOEP), Palladium(II) octaethylporphine (PdOEP), Platinum(II)-5,10,15,20-tetrakis-(2,3,4,5,6-pentafluorphenyl)-porphyrin (PtTfPP), palladium(II)-5,10,15,20-tetrakis-(2,3,4,5,6-pentafluorphenyl)-porphyrin (PdTFPP), platinum(II) octaethylporphyrinketone (PtOEPK), palladium(II) octaethylporphyrinketone (PdOEPK), platinum(II) tetraphenyltetrabenzoporphyrin, (PtTPTBP), meso-Tetraphenyl-tetrabenzoporphine Palladium Complex (PdTPTBP), platinum(II) tetraphenyltetranaphthoporphyrin (PtTPTNP), and palladium(II) tetraphenyltetranaphthoporphyrin (PdTPTNP).

45. The method of one of claims 10-44, wherein the oxygen-sensitive fluorophore is a is a porphyrin metal complex.

46. The method of claim 45, wherein the oxygen-sensitive fluorophore is PtTfPP.

47. The method of one of claims 1-9, wherein the sample is a liquid sample.

48. The method of one of claims 1-7, wherein the sample comprises cells.

49. The method of claim 48, wherein the sample comprises cell media.

50. The method of claim 1, wherein the suspension is contained within a microwell.

51. The method of claim 2, wherein the oxygen-sensitive composition is adhered to a surface of a microwell.

52. The method of claim 51, wherein the oxygen-sensitive composition is adhered to the bottom of a microwell.

53. The method of claim 6, wherein the hydrogel is contained within a microwell.

54. The method of claim 6, wherein forming a hydrogel comprises exposure to temperature, pH, ions, light, or a small molecule inducer.

55. The method of one of claims 50-54, wherein the bottom of the microwell is transparent.

56. The method of one of claims 1-9, wherein determining the level of oxygen in the sample comprises comparing the light output to one or more reference values.

57. The method of claim 56, wherein one of the reference values is obtained from light output of a deoxygenated standard.

58. The method of claim 56, wherein one of the reference values is obtained from light output of an oxygenated standard.

59. The method of claim 56, wherein one or more of the reference values is obtained from light output of standards of varying degrees of oxygenation.

60. The method of one of claims 1-9, wherein steps (b)-(d) are repeated to monitor the level of oxygen over time.

61. The method of claim 1, further comprising:
(i) placing the oxygen-sensitive composition into a standard of known oxygenation level to form a suspension of the composition within the standard;
(ii) exposing the oxygen-sensitive composition within the standard to light within an excitation spectrum of the oxygen-sensitive composition;
(iii) detecting light output within an emission spectrum from the oxygen-sensitive composition within the standard; and
(iv) determining a reference value for the light output from the oxygen-sensitive composition within the standard.

62. The method of claim 61, wherein steps (b)-(d) and (i)-(iv) are repeated to monitor the level of oxygen over time.

63. The method of claim 2, further comprising:
(i) contacting the oxygen-sensitive composition with a standard of known oxygenation level;
(ii) exposing the oxygen-sensitive composition within the standard to light within an excitation spectrum of the oxygen-sensitive composition;
(iii) detecting light output within an emission spectrum from the oxygen-sensitive composition within the standard; and
(iv) determining a reference value for the light output from the oxygen-sensitive composition within the standard.

64. The method of claim 63, wherein steps (b)-(d) and (i)-(iv) are repeated to monitor the level of oxygen over time.

65. The method of claim 6, further comprising:
(i) forming a standard hydrogel comprising the oxygen-sensitive composition and a standard of known oxygenation level;
(ii) exposing the oxygen-sensitive composition within the standard hydrogel to light within an excitation spectrum of the oxygen-sensitive composition;
(iii) detecting light output within an emission spectrum from the oxygen-sensitive composition within the standard hydrogel; and
(iv) determining a reference value for the light output from the oxygen-sensitive composition within the standard hydrogel.

66. The method of claim 65, wherein steps (b)-(d) and (i)-(iv) are repeated to monitor the level of oxygen over time.

67. The method of claim 8, wherein measuring fluorescence intensities comprises:
(i) exposing the oxygen-sensitive composition to light within an excitation spectrum of the oxygen-sensitive composition;
(ii) detecting light output within an emission spectrum from the oxygen-sensitive composition.

68. The method of claim 8, wherein fluorescence intensities are measured over a time period of 1 minute to 1 week.

69. The method of claim 68, wherein fluorescence intensities are measured every 1 second to 1 hour over the time period.

70. The method of claim 8, wherein, prior to step (a), the sample(s) are administered an agent of interest for a specified duration of time of 1 minute to 1 week.

71. The method of claim 8, wherein, prior to step (b), the sample(s) are administered an agent that prevents mitochondrial activity, such that the oxygen consumption rate measured is the result of non-mitochondrial cellular respiration.

72. The method of claim 71, wherein the agent that prevents mitochondrial activity is rotenone, oligomycin, antimycin, or combination.

73. The method of claim 8, wherein, prior to step (b), the sample(s) are administered an agent that stimulates the respiration, and the oxygen consumption rate is measured.

74. The method of claim 73, wherein the agent that stimulates the respiratory chain is carbonyl cyanide-p-trifluoromethoxyphenylhydrazone (FCCP).

75. The method of claim 8, wherein, prior to step (b), the sample(s) are administered an agent that inhibits ATP production, and the oxygen consumption rate is measured.

76. The method of claim 75, wherein the agent that stimulates the respiratory chain is oligomycin.

77. A method to measure basal mitochondrial respiration, comprising:
(i) measuring basal cellular respiration by contacting the sample(s) with an oxygen-sensitive composition;
(ii) measuring non-mitochondrial cellular respiration by contacting the sample(s) with an oxygen-sensitive composition and an agent or combination that prevents mitochondrial activity; and
(iii) calculating basal mitochondrial respiration by subtracting non-mitochondrial oxygen consumption from basal cellular oxygen consumption.

78. A method to measure maximal mitochondrial respiratory capacity, comprising
(i) measuring non-mitochondrial cellular respiration by contacting the sample(s) with an oxygen-sensitive composition and an agent or combination that prevents mitochondrial activity;
(ii) further measuring the maximum cellular oxygen consumption by contacting the sample(s) with an agent that stimulates mitochondria respiration;
(iii) calculating maximal mitochondrial respiratory capacity by subtracting non-mitochondrial cellular oxygen consumption from the maximum cellular oxygen consumption.

79. A method to measure spare respiratory capacity, comprising:
(i) measuring basal cellular respiration by contacting the sample(s) with an oxygen-sensitive composition;
(ii) measuring the maximum cellular oxygen consumption by contacting the sample(s) with an agent that stimulates mitochondria respiration; and
(iii) calculating spare mitochondrial respiratory capacity by subtracting basal cellular oxygen consumption from the maximum cellular oxygen consumption.

80. The method of claim 70, where the samples are administered an agent of interest for a specified amount of time.

81. The method of claim 80, further comprising: measuring basal cellular respiration by contacting the sample(s) with an oxygen-sensitive composition; measuring non-mitochondrial cellular respiration by contacting the sample(s) with an oxygen-sensitive composition and an agent or combination that prevents mitochondrial activity; measuring the maximum oxygen consumption by contacting the sample(s) with an agent that stimulates mitochondria respiration; and calculating metabolic potential by subtracting non-mitochondrial oxygen consumption from both the maximum oxygen consumption and the basal cellular respiration, then taking the ratio of these values.

82. A method to measure mitochondrial respiration linked to ATP production, comprising:
(i) measuring basal cellular respiration by contacting the sample(s) with an oxygen-sensitive composition;
(ii) measuring respiration linked to proton leak by contacting the sample(s) with an oxygen-sensitive composition and an agent or combination that prevents mitochondrial ATP production; and
(iii) calculating mitochondrial respiration linked to ATP production by subtracting respiration linked to proton leak from basal cellular oxygen consumption.

83. A method to measure mitochondrial coupling efficiency, comprising:
(i) measuring basal cellular respiration by contacting the sample(s) with an oxygen-sensitive composition;
(ii) measuring respiration linked to proton leak by contacting the sample(s) with an oxygen-sensitive composition and an agent or combination that prevents mitochondrial ATP production;
iii) calculating mitochondrial respiration linked to ATP production by subtracting respiration linked to proton leak from basal cellular oxygen consumption; and
(iv) calculating coupling efficiency by taking the ratio of respiration linked to ATP production to basal cellular oxygen consumption.

84. The method of one of claims 1-83, wherein the sample comprises T cells, B cells, or NK cells.

85. The method of one of claims 1-83, wherein the sample comprises embryonic stem cells, tissue-specific stem cells, mesenchymal stem cells, or induced pluripotent stem cells.

86. The method of one of claims 1-83, wherein the sample comprises adherent cell lines.

87. The method of one of claims 1-83, wherein the sample comprises suspension cell lines.

88. The method of any one of claims 1-83, wherein the sample comprises bacterial cells.

89. An oxygen detection system comprising:
(a) an oxygen-sensitive composition comprising an oxygen-sensitive fluorophore and a substrate;
(b) a liquid sample comprising cells in an assay media;
(c) an oxygenated standard; and
(d) a deoxygenated standard.

90. The oxygen detection system of claim 89, wherein the oxygenated standard comprises the assay media in the absence of cells.

91. The oxygen detection system of claim 89, wherein the deoxygenated standard comprises glucose oxidase and glucose.

92. The oxygen detection system of claim 89, wherein the deoxygenated standard comprises Na₂SO₃ and water.

93. The oxygen detection system of claim 89, further comprising a microwell plate, wherein at least a first well of the microwell plate comprises the liquid sample and the oxygen-sensitive composition, at least a second well of the microwell plate comprises the oxygenated standard and the oxygen-sensitive composition, and at least a third well of the microwell plate comprises the deoxygenated standard and the oxygen-sensitive composition.

94. The oxygen detection system of claim 93, wherein the oxygen-sensitive compositions are adhered to a surface of the wells of the microwell plate.

95. The oxygen detection system of claim 94, wherein the oxygen-sensitive compositions are adhered to the bottom of the wells of the microwell plate.

96. The oxygen detection system of claim 95, wherein the oxygen-sensitive composition is suspended in the liquid sample, the oxygenated standard, and/or the deoxygenated standard.

97. The oxygen detection system of claim 83, wherein the oxygen-sensitive composition is suspended in a hydrogel with the liquid sample, the oxygenated standard, and/or the deoxygenated standard.

98. The oxygen detection system of claim 89, further comprising an instrument capable of detecting fluorescence.

99. A method to measure an oxygen consumption rate for a population of cells, comprising:
(a) contacting an oxygen-free control, an oxygen-saturated control, and one or more test samples comprising the cells with an oxygen-sensitive composition;
(b) measuring the basal respiration rate of the cells by:
(i) measuring fluorescence intensities of the oxygen-sensitive composition in the controls and samples(s) over a period of time;
(ii) converting the fluorescent intensity of the sample(s) to oxygen concentration using (i) the stern-volume equation and (ii) the fluorescent intensities of the controls;
(c) contacting the controls and samples(s) with oligomycin and measuring the non-mitochondrial respiration rate of the cells by:
(i) measuring fluorescence intensities of the oxygen-sensitive composition in the controls and samples(s) over a period of time;
(ii) converting the fluorescent intensity of the sample(s) to oxygen concentration using (i) the stern-volume equation and (ii) the fluorescent intensities of the controls;
(d) contacting the controls and samples(s) with FCCP and measuring the maximum oxygen consumption of the cells by:
(i) measuring fluorescence intensities of the oxygen-sensitive composition in the controls and samples(s) over a period of time;
(ii) converting the fluorescent intensity of the sample(s) to oxygen concentration using (i) the stern-volume equation and (ii) the fluorescent intensities of the controls; and
(e) contacting the controls and samples(s) with rotenone and/or antimycin and the oxygen consumption rate in the absence of mitochondrial activity by:
(i) measuring fluorescence intensities of the oxygen-sensitive composition in the controls and samples(s) over a period of time;
(ii) converting the fluorescent intensity of the sample(s) to oxygen concentration using (i) the stern-volume equation and (ii) the fluorescent intensities of the controls.

100. The method of claim 99, further comprising calculating the basal mitochondrial respiration rate by subtracting the oxygen consumption rate in the absence of mitochondrial activity from the basal respiration rate.

101. The method of claim 99, further comprising calculating spare respiratory capacity by subtracting the basal mitochondrial respiration rate from the maximum oxygen consumption.

102. The method of claim 99, further comprising calculating maximum respiration by subtracting the non-mitochondrial respiration rate or oxygen consumption rate in the absence of mitochondrial activity from the maximum oxygen consumption.

103. The method of claim 99, further comprising calculating metabolic potential by dividing maximum respiration by basal mitochondrial respiration.

104. The method of claim 99, wherein steps (a) through (e) are performed in order.
